(19) United States
(12) Patent Application Publication
(10) Pub. No.: US 2021/0138268 A1
(43) Pub. Date: May 13, 2021

(54) ARCHIVING RADIATION THERAPY RECORDS FOR TRANSMISSION AND REVIEW

(71) Applicant: THE BOARD OF REGENTS OF THE UNIVERSITY OF TEXAS SYSTEM, Austin, TX (US)

(72) Inventors: Yulong YAN, Plano, TX (US); Robert TIMMERMAN, Westlake, TX (US)

(73) Assignee: THE BOARD OF REGENTS OF THE UNIVERSITY OF TEXAS SYSTEM, Austin, TX (US)

(21) Appl. No.: 17/095,179

(22) Filed: Nov. 11, 2020

Related U.S. Application Data
(60) Provisional application No. 62/933,950, filed on Nov. 11, 2019.

(51) Int. Cl. A61N 5/10; G16H 40/20; G16H 20/40; G16H 70/20; G16H 30/20; G16H 50/20; G16H 40/40; G16H 50/50; G16H 50/70; G06F 16/23

(52) U.S. Cl. CPC A61N 5/1038; G16H 40/20; G16H 20/40; G16H 70/20; G16H 30/20; A61N 2005/1041; G16H 40/40; G16H 50/50; G16H 50/70; G06F 16/2365; G16H 50/20

(57) ABSTRACT

Disclosed embodiments include systems and methods for archiving complete radiation records for transmission and review. In various embodiments, the radiation therapy archiving system miniaturizes some of the essential features of radiation therapy planning systems including radiation therapy data reading and writing capabilities, functionality for representing contoured objects and other radiation therapy specific features, and functionality for viewing radiation therapy plans. Compact radiation therapy datasets generated by the radiation therapy archiving system may accurately represent previous radiation doses administered to a patient. To help patients transition from one radiation therapy clinic to a remote clinic, radiation therapy datasets may be downloaded to a personal computer and transferred a remote treatment planning system to be used during the design phase of ongoing radiation therapy treatments.

### CROSS REFERENCE TO RELATED APPLICATIONS

This application claims the benefit of U.S. Provisional Application Ser. No. 62/933,950 filed Nov. 11, 2019, the entire contents of which is hereby incorporated by reference.

### FIELD OF DISCLOSURE

The present disclosure relates generally to data processing and visualization methods for medical applications, and, more specifically, to archiving complete radiation therapy plan datasets and records for transmission and review.

### BACKGROUND

Radiation therapy facilities, such as hospitals and oncology clinics, keep detailed records of radiation doses administered to patients to serve as a reference regarding previously delivered radiation doses. This historical radiation treatment record provides important insight to better understand changes in patient status (e.g., a new deficit or toxicity) and/or the occurrence of new tumors proximate to old treatment sites. Additionally, toxicity levels for radiation depend on the cumulative sum of radiation absorbed from current and previous treatments. Therefore, it is important that patients retain comprehensive radiation therapy records and reproduce complete prior radiation dose distributions to their new provider if they begin receiving treatment at a new radiation therapy facility.

To prescribe a given course of radiotherapy care, typically CT images of the region to be treated are obtained just prior to the actual radiotherapy course and serve as the “reference” image data set. These reference images alone are stored in a standardized image format called DICOM. As a part of the treatment planning process, in a separate computer program that can read in DICOM reference images, the treating physicians or dosimetrists electronically draw lines around certain structures manually or using automated tools. These lines around identified structures are called “contours” and each structure so identified falls into two categories: 1. Targets, and 2. Normal structures, also known as organs at risk (OARs). The next step in the radiotherapy planning process is to model and then calculate the dose build-up from beams of radiation incident upon the patient and aimed at the target(s). Dose, location, build-up, and other beam information is typically referred to as the “RT plan”. The construction of this dose build-up is simulated by the planning system and viewed on the display. The RT plan, however, contains sufficient detail so that it can actually be carried out in a patient if the radiation delivery machines are calibrated and validated to the representations of the RT plan in the treatment planning computer. Therefore, constructing an RT plan is a comprehensive process that requires considerable computing power and sophisticated software.

Radiotherapy treatment planning software stores the representation of the treatment in a standard format called DICOM-RT. DICOM-RT is radiation therapy specific subset of DICOM modalities that model radiation therapy clinical practice. DICOM-RT includes the original DICOM reference images that are correlated to the contours/structures, the RT plan (including beam information), and a dose matrix generated by the planning system. DICOM-RT also includes fields for information referred to as “RT records” that document the actual dose delivered to the patient over the entire course of the therapy using the treatment plan. Data entry into the record locations for DICOM-RT, however, is not essential to simply view the treatment plan. Typically, one planning image element and five of DICOM-RT elements (e.g., a RT Structure Set, RT Plan, RT Image, RT Dose, and RT Record) are included in each plan dataset. The planning image and DICOM-RT elements are referred to as the plan “objects.” More elements, such as fusion image sets, image registration, and image segmentation objects, could also be included in a more complicated plan dataset when multi-modality image sets are utilized in the planning process. Simple PDF-like distilled representations of this DICOM-RT information are commonly created to include in the patient's medical record. This PDF information, however, is rudimentary and lacks the ability to review dose build-up throughout the entire reference images. Currently, reviewing the entire plan dataset including all DICOM-RT elements requires using a sophisticated, processing intensive radiotherapy treatment planning software that can only be run on specialized hardware including a powerful graphics processor and vast amounts of memory resources (e.g., random access memory (RAM)). Therefore, it is desirable, to have a system that can achieve miniaturized versions of comprehensive plan datasets including reference image and DICOM-RT elements for display on a regular computer.

### SUMMARY

In one aspect, disclosed herein are methods for archiving radiation therapy records for transmission and review comprising: receiving a plurality of radiation therapy objects; parsing the plurality of radiation therapy objects to articulate a radiation therapy dataset; generating a directory object including a list of components included in the radiation therapy dataset; embedding a viewer with the radiation therapy dataset, the viewer navigating the directory object to display one or more radiation therapy records included in the radiation therapy dataset; and writing the radiation therapy dataset, the itemized directory object, and the viewer to a portable storage medium.

In one aspect, the radiation therapy dataset comprises a radiation therapy plan including one or more plan objects that visualize one or more aspects of a radiation therapy treatment. In one aspect, the plan objects include radiation beam delivery plans, dose plans, dose distribution profiles, and dose statistics.

In one aspect, the method comprises parsing the plurality of radiation therapy objects by: analyzing a hierarchical network of links to locate the plurality of radiation therapy objects having viewing data included in the radiation therapy dataset; for each radiation therapy object included in the plurality of radiation therapy objects, miniaturizing the radiation therapy object by separating the viewing data from plan creation data included in the radiation therapy object; downloading the viewing data as the one or more plan objects; and organizing the one or more plan objects as the radiation therapy dataset.

In one aspect, viewing data includes image data, contour data, delivery beam data, radiation dose data, and other data required to display the one or more radiation therapy records. In one aspect, plan creation data includes treatment simulation physics and modeling algorithms, radiation delivery machine-specific physics data and models, computational algorithm modules for creating radiation therapy plans, and intermediate files used in the process of making radiation therapy plans.

In one aspect, the hierarchical network of links comprises: patient IDs, study IDs, series IDs, and image IDs. In one aspect, the viewer comprises a miniaturized executable file having a few dynamically loadable graphics libraries. In one aspect, the viewer presents the radiation therapy datasets in a hierarchy that is defined in DICOM standards for radiation therapy practice.

In one aspect, the viewer navigates the itemized directory object to display one or more radiation therapy records by: parsing the directory object to aggregate the one or more radiation therapy objects included in each plan object, wherein the directory object includes a network of external links between the one or more radiation therapy objects and the one or more plan objects; checking data integrity of the radiation therapy dataset by verifying every radiation therapy object required visualize the one or more plan objects is included in the radiation therapy dataset; and rendering, by the viewer, the radiation therapy plan object using one or more graphics libraries.

In one aspect, the directory object further comprises a network of internal links between the components of the radiation therapy dataset included in each radiation therapy plan object and the one or more plan objects. In one aspect, the viewer renders the radiation therapy plan object on a laptop, smartphone, tablet, or other personal computer device.

In one aspect, disclosed herein are methods for planning radiation therapy treatments comprising: receiving a radiation therapy dataset and a viewer from a portable storage medium, wherein the radiation therapy dataset includes a radiation therapy plan having one or more plan objects that visualize one or more aspects of a radiation therapy treatment; displaying, by the viewer, the one or more plan objects, wherein the one or more plan objects include treatment planning information; evaluating the treatment planning information; importing the treatment planning information into a treatment planning system; and generating a new radiation therapy plan including one or more new radiation therapy treatments using the treatment planning information.

In one aspect, the one or more plan objects comprise planning images, segmented structure sets, 3D dose objects, and radiation therapy plan objects. In one aspect, treatment planning information comprises image data, contour data, dose data, and delivery device data collected for the radiation therapy treatment plan.

In one aspect, the evaluating the treatment planning information comprises: viewing a 3D dose object including dose distributions and segmented structure sets displayed on a planning image, wherein the segmented structure sets include one or more treated target areas and nearby normal structures; generating dose statistics of the treated target areas and the nearby normal structures; and determining a patient's radiation exposure based on the dose statistics.

In one aspect, the dose statistics include isodose lines describing an amount of radiation absorbed by the treated target areas and the nearby normal structures within the isodose lines. In one aspect, the method comprises: transmitting the new radiation therapy plan to a radiation delivery device to administer the new radiation therapy treatment to a patient. In one aspect, the new radiation therapy plan comprises: radiation dose prescriptions, plan delivery parameters, and instructions for operating a radiation treatment delivery device.

In one aspect, disclosed herein are radiation therapy archiving systems comprising: a miniaturization agent receiving radiation therapy objects from an object database, wherein the miniaturization agent includes parsing logic and a coder/decoder for generating complete radiation therapy datasets, the parsing logic parsing the radiation therapy objects to articulate a complete radiation therapy dataset including one or more plan objects that visualize one or more aspects of a radiation therapy treatment, and the coder/decoder formatting radiation therapy objects for processing by the parsing logic and standardizing the format of the one or more plan objects in a hierarchy that is defined in DICOM standards for radiation therapy practice; a radiation therapy dataset database storing the complete radiation therapy datasets generated by the miniaturization agent; a miniature viewer including a miniaturized executable file having one or more dynamically loadable files that display the one or more plan objects included in the radiation therapy dataset; and an archiving module generating a directory object including a network of links associating aspects of the radiation therapy objects included in the one or more plan objects, wherein the archiving module writes the directory object, the radiation therapy dataset, and the miniature viewer to a portable display medium.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Various objectives, features, and advantages of the disclosed subject matter can be more fully appreciated with reference to the following detailed description of the disclosed subject matter when considered in connection with the following drawings, in which like reference numerals identify like elements.

FIG. 1 depicts an exemplary system for archiving compact, viewable RT datasets, according to embodiments of the disclosure.

FIG. 2A illustrates an exemplary network of links used to navigate between objects and components of objects included in an RT dataset, according to embodiments of the disclosure.

FIG. 2B illustrates an exemplary beam plan, according to embodiments of the disclosure.

FIG. 2C illustrates an exemplary dose plan, according to embodiments of the disclosure.

FIGS. 2D-2E illustrate two exemplary presentations of dose statistics for an RT treatment, according to embodiments of the disclosure.

FIG. 3 is a flow diagram illustrating an exemplary process for generating compact, viewable RT datasets, according to embodiments of the disclosure.

FIG. 4 is a flow diagram illustrating showing an exemplary process for importing archived treatment planning information into a treatment planning system, according to embodiments of the disclosure.

FIG. 5 is a block diagram of an illustrative computer device that may be used to implement the system of FIG. 1, according to embodiments of the disclosure.

### DETAILED DESCRIPTION

Unlike the majority of imaging modalities that DICOM standards were originally made for (e.g., Computed Tomography (CT), Magnetic Resonance Imaging (MRI), and the like), radiation therapy (RT) needs multiple modalities to model the clinical practice. Existing radiation therapy records are stored in DICOM objects of multiple modalities. Many of these DICOM objects are very large files. For each patient, many internal and external links between different DICOM object modalities (e.g., reference images and contours) are used to articulate hundreds of DICOM objects into complete radiation therapy datasets. These bulky datasets are difficult to manipulate and easily corrupted. Therefore, even performing basic functions (viewing, editing, transferring, etc.) with radiation therapy records requires significant storage capacity, memory allocation, and processing resources. To avoid complications with large files, most patients resort to crude processes for transferring radiation records such as capturing screen views, printing hard copy reports, or obtaining a digital copy of disparate information for each DICOM object modality in a limited PDF format. Failure to obtain comprehensive radiation therapy records, limits the ability of the receiving center to develop an optimized radiation plan and puts patients in danger of over exposure to radiation.

The RT achieving system of the disclosure improves upon existing tools for storing and transferring RT treatment plans by generating compact RT datasets including miniature versions of DICOM-RT objects. The miniature RT objects are compact so that they can be viewed using a personal computer, tablet, or mobile device. The compact RT datasets and miniature RT objects store a complete record of the RT treatment data so they can be used to design a new treatment plan. For example, the contour and normal structure information in the miniature RT objects can be used to identify changes in the size of existing tumors and the appearance of new tumors in the patient. The dose information included in the compact RT datasets and miniature RT objects may also be used to determine the aggregate radiation exposure (i.e., dose build-up) of tissue structures located at and around prior radiation cites. The compact RT datasets and miniature RT objects are also compatible with any radiation treatment planning system so they may be readily transferred to another system and or provider without risk of data loss or corruption.

The RT achieving system also includes a viewer that may be used to display any RT treatment records. At run time, the viewer reads in the RT datasets from common, portable storage devices such as a CD or flash drive. Once the patient's RT dataset from a previous treatment is imported, the viewer displays the treatment plan in its entirety, irrespective of what kind of treatment planning and delivery platforms were actually used to plan and deliver the dose or the number of DICOM objects included in the dataset. This standalone system for archiving and viewing comprehensive DICOM-RT datasets allows patients, physicians, dosimetrists, and the like to understand whether the previous radiotherapy impacted any current, ongoing changes in the patient's health or contemplate whether additional radiation might be delivered to the patient for new problems such as tumor progression. The system may provide this viewing capability to commonly available computers such as desktops and laptops by eliminating the requirement for specialized computer hardware and/or workstations of most treatment planning systems. Finally, to streamline planning of new radiation treatments, the RT dataset displayed by the system could be downloaded onto the center's treatment planning system to be incorporated or accounted for when designing a new course of radiotherapy.

In various embodiments, the system may include only the viewing functions of a conventional radiotherapy treatment planning system without the algorithms used for contour generation, beam generation, dose calculation, or validation. The viewing functions may be optimized and miniaturized so that the resultant compiled, executable program files could reside on the same common, portable storage device (CD or flash drive) as the patient's DICOM-RT files.

Radiation therapy (RT) treatment workflows are complicated processes that should be executed effectively to avoid serious complications due to toxic levels of absorbed radiation. A typical RT treatment workflow involves designing an RT plan, prescribing an RT treatment based on the RT plan, and providing RT treatment. The design phase is a data intensive process that searches the optimal set of machine parameters that make the delivery machine deposit the exact amount of dose to targeted 3D volume. The quantitative dose distribution will be overlaid over high resolution images for physicians to review plan quality. To prescribe a radiation dose, physicians review RT plan datasets to understand how a prescribed radiation dose will interact with an identified tumor site and the surrounding tissue. The RT plan datasets incorporate a plurality of distinct RT objects having one or more different object modalities. The RT objects may be organized according to a network of internal links between different components of an object and external links between different objects. By navigating these networks of internal and external links, objects required to visualize one or more aspects of an RT dataset may be located, organized, and/or assembled to provide comprehensive radiation treatment information. For example, reference image and contour objects may be used to identify anatomical groupings of tumor sites and healthy structures; RT plan objects may be used to simulate the route(s) of one or more radiation beams incident on the patient to optimize radiation delivery to the tumor sites and minimize exposure elsewhere; dose matrices and other dose plan objects may describe radiation dose build-up that occurs when radiation is delivered to each anatomical grouping or other structure identified in the contour object; and dose record objects may describe actual dose amounts administered and/or parameters used to administer the dose (e.g., machine calibration, patient position, delivery device used, shielding, time, provider, and the like).

To make RT more efficient and accurate, treatment planning systems have automated much of the RT treatment design and planning process. Treatment planning systems may reduce the amount of time needed to design an RT treatment and provide more accurate beam routes. The amount of data, the disparate data sources, and the interdependence of the disparate data required by treatment planning systems, however, makes RT records bulky, untransferable, and subject to disintegration if data from one or more sources is lost or unavailable.

Embodiments of the present disclosure may improve the effectiveness and safety of radiation therapy (RT) by archiving comprehensive RT datasets in a compact, transferable format and providing functionality for downloading, parsing, and viewing previously generated RT plans on a personal computer (desktop, laptop, mobile phone, and the like) or other remote computer. The record archiving system can include a miniaturization agent that may reduce the memory, storage, and/or processing capacity needed to read, write, and/or organize RT objects and/or articulate RT datasets from a plurality of RT objects. A compact, self-sufficient RT viewer is also provided to enable high resolution visualization depicting planning information, contours, beam routes, and or other visual aspects of RT datasets to be reviewed on a personal computer or other remote computer.

In various embodiments, RT datasets may include many different RT object modalities that describe various aspects of RT treatment workflows. RT object classes may be specific to RT treatments and/or included in other medical records. In various embodiments, RT objects may include reference images captured by a medical imaging device (e.g., CT scanner, magnetic resonance (MR) imager, PET scanner, fluoroscope, etc.). Specific reference images used to design RT plans can include image sets comprising slice by slice representations of human anatomy. The slices may depict patient tissue at a sequence of incrementally spaced depth distances along one or more orientation axes (e.g., axial, sagittal, coronal). Image attributes (e.g., contrast, bolus, field of view, resolution, etc.) may also be included in RT reference image objects. The reference image may have one or more external links to one or more other RT object modalities, for example, record objects that may include attributes (e.g., height, weight, age, race, gender, family medical history, medications, etc.) and/or medical history of the patient associated with the reference image. Reference images may also be linked to contour objects, planning objects, and dose objects, that include one or more visual components overlaid on the reference image.

Additional RT object modalities can include RT Structure Sets, RT Plans, RT Images, RT Doses, and RT Records. RT Plan objects may include dose prescription attributes (e.g., total dose, number of dose fractions, dose objectives, and the like), delivery device configurations (e.g., collimator rotation, treatment modality, RT system model, gantry, beam intensity, collimator parameters, patient positioning instructions, etc.), navigation instructions describing the route and any filtering and/or shaping modifications for each beam administering radiation, and/or patient setup information (e.g., field of interest, volume of interest, source to skin distance, etc.). RT Structure Set objects can include groupings of anatomical structures (e.g., normal structures, organs at risk, tumors, etc.) and regions of interest (e.g., tumor volume) as well as contours for segmenting structure sets by texture (e.g., color, color intensity, pattern, etc.). RT Dose objects can include dose build-up information including volumetric dose distribution and dose volume histograms. RT Record objects can include nominal doses administered to patients, beam session records describing the dose (e.g., in monitor units) delivered to each radiation field at each treatment session, the date of the RT treatment, RT system configurations (e.g., collimator rotation, treatment modality, RT system model, gantry, etc.) and/or patient setup information (e.g., field of interest, volume of interest, source to skin distance, etc.).

Information from different RT object modalities may be combined to create RT datasets used to simulate radiation treatments and program delivery devices to administer radiation to patients. In various embodiments, different RT object modalities may be combined to generate one or more datasets and/or visualizations used during RT planning. For example, RT Structure Set objects (e.g., groupings of healthy tissues, tumors, organs at risk, etc.) may be combined with RT reference images to generate structure datasets including visualizations segmenting portions of reference images into distinct anatomical groupings. In various embodiments, visualizations included in RT datasets that combine RT Structure Set and RT reference image objects may resemble contour maps overlaying color coding and/or other distinguishing textures over areas of interest and/or structure sets shown in a reference image (e.g., a CT scan image).

In various embodiments, RT Plan objects may be combined with reference images and/or structure set objects to generate dose statistics datasets including dose volume histograms and other dose distribution profiles showing radiation dose data superimposed on a reference image (e.g., a CT scan image) of an area of interest. RT Plan objects including dose prescription attributes from two or more treatments may be combined to generate historical dose statistics datasets including historical dose volume histograms and other historical dose distribution data that tracks the aggregate accumulation of radiation in one or more structures. RT Plan objects may also be combined with contour objects and reference images to generate beam plan datasets including maps that illustrate beam routes and contours superimposed over a reference image (e.g., CT scan).

In various embodiments, to generate RT datasets, different object modalities may be combined using a network of internal and external links. The network of links can be used to determine the objects and the components within each objected needed to assemble an image, visualization, and/or dataset. The network of links may define a hierarchy for an object and/or series of objects. For example, one or more objects may be combined to generate an image or visualization. Each individual image may be included in a group of related images called a series. One or more series of images may be included in a study and each patient may have several studies included in their complete RT data set. Navigating this network of links allows the objects included in each image, series, study, and/or patient of interest to be efficiently accessed and downloaded. FIG. 2A below illustrates an exemplary network of links uses to associated objects and object components.

RT datasets (e.g., structure sets, RT plans, dose datasets, and beam plan datasets, navigation datasets, and treatment records, and the like) are large files that are difficult to review, transfer, edit, and otherwise manipulate. RT datasets may be assembled from a plurality of objects having different object modalities. The RT objects may be connected through a network of links to internal and external information. For example, internal links between different components of the same object and external links between different objects. The volume of data included in RT datasets combined with the network of links between different RT objects within the dataset makes RT datasets difficult to interpret, export, and transfer. Additionally, if one or more data sources contributing information to an RT dataset is lost, moved, destroyed, and/or unavailable the entire RT dataset is corrupted and may not be viewed or analyzed.

In various embodiments, the RT archiving system generates a compact version of RT datasets (i.e., miniature RT objects) that are writable to a portable storable medium (e.g., CD) and viewable on a personal computer. The RT archiving system may reduce the amount of storage, memory, and processing capacity needed to display and manipulate RT datasets by only incorporating plan review components of a RT planning system and leaving out bulky files for creating, planning, and/or simulating RT plans. The extracted features and algorithms are then optimized for efficient display on personal computers. By reducing the size and complexity of RT datasets, the RT archiving system may make converting and encrypting RT datasets easier, thereby increasing the security of patient RT data. Furthermore, smaller RT objects and datasets (i.e., the miniature RT objects) are less vulnerable to data corruption that may occur when a system fails while performing processing intensive read/write operations to and/or from memory and/or storage or encoding/decoding operations converting RT datasets and/or objects from one format to another format (e.g., from a raw image format, .png, .jpeg, etc. to Digital Imaging and Communication in Medicine (DICOM) RT standard format).

In various embodiments, the RT archiving system may provide a compact, self-sufficient viewer having functionality for displaying RT datasets on a personal computer or other remote computer. The viewer may also include functionality for manipulating RT planning information included in RT datasets using a personal computer or remote computer. In various embodiments, the viewer may be miniaturized into a compact executable form that may download, display, edit, retrieve, write, re-format, or otherwise manipulate RT datasets. By articulating compact RT datasets and miniaturizing the viewer for displaying one or more visual representations of the compact dataset, the RT archiving system makes transferring comprehensive RT datasets easier. The viewer and compact RT datasets (i.e., the miniature RT objects) described herein may be written to a portable storage medium and easily transferred by any treatment planning system or personal computer. Archiving compact RT datasets together with a self-sufficient viewer allows patients to transfer digital, comprehensive, undistorted, and interpretable RT datasets assembled from objects of different modalities to remote providers and display the RT datasets on a remote computer so they may be reviewed by physicians, patients, and dosimetrists. Using the miniaturized viewer, new providers can rapidly load and view the most up to date RT datasets for a patient to expedite the RT treatment planning phase and minimize risks of radiation toxicity.

FIG. 1 depicts an exemplary system 100 for archiving RT datasets for transmission and review. In various embodiments, the system 100 may facilitate transfer of RT datasets and other patient records to remote facilities by digesting RT objects from a treatment planning system of a patient's previous RT provider; parsing and articulating RT objects into complete RT datasets; generating an itemized directory object for organizing RT objects, object components, and other aspects of RT datasets, embedding a miniature viewer with the RT datasets to create compact, viewable RT datasets (i.e., miniature RT objects) that may be written to a portable storage medium; and transferring viewable RT datasets to a treatment planning system that may access archived planning information included in the miniature RT objects to generate new RT plans describing ongoing RT treatments to be administered by a treatment delivery device.

As shown in FIG. 1, the RT archiving system 110 may include various components, for example, an RT object database 102, a miniaturization agent 111, a RT datasets database 114, a miniature viewer 115, and an archiving module 118. As used herein, the term “component” may be understood to refer to computer executable software, firmware, hardware, and/or various combinations thereof. It is noted that where a component is a software and/or firmware component, the component is configured to affect the hardware elements of an associated system. It is further noted that the components shown and described herein are intended as examples. The components may be combined, integrated, separated, or duplicated to support various applications. Also, a function described herein as being performed at a particular component may be performed at one or more other components and by one or more other devices instead of or in addition to the function performed at the particular component. Further, the components may be implemented across multiple devices or other components local or remote to one another. Additionally, the components may be moved from one device and added to another device or may be included in both devices.

As shown in FIG. 1, an exemplary system 100 may include an RT object database 102. In various embodiments the RT object database 102 can be included in a treatment planning system operated by radiology center or other clinic administering RT to the patient. The RT archiving system 110 may import RT objects included in the RT object database 102 from a treatment planning system using any known wired or wireless communications path. In various embodiments, the RT archiving system 110 may be implemented as an independent application installed on a personal computer 150 or treatment planning system 130. The record archiving system 110 may also be integrated with a treatment planning system 130.

The RT objects database 102 may include RT objects of different modalities (e.g., RT Images, RT Structure sets, RT Plans, RT Doses, RT Records, and the like) exported from a treatment planning system. In various embodiments, the RT objects database 102 may store RT objects generated from previously planned and/or administered treatments. RT objects included in the RT objects database 102 may be collected and maintained on a treatment planning system operated by the patient's RT treatment provider. The RT objects database 102 may store RT objects in various ways including, for example, as a flat file, indexed file, hierarchical database, relational database, unstructured database, graph database, object database, and/or any other storage mechanism.

The RT objects database 102 may provide RT objects to the RT archiving system 110 through an automated transfer process (e.g., wired or wireless transfer of digital RT object information) and/or a manual transfer process (e.g., download RT objects to a portable hard drive or other storage medium, connect the hard drive to the RT archiving system 110, and download the RT objects onto a computer implementing the RT archiving system 110). In various embodiments, a selection of RT objects (e.g., RT objects associated with a particular patient, RT treatment, RT clinic, prescribing physician, etc.) are provided to the RT archiving system 110 for further processing. The RT archiving system 110 may also search the RT objects database 102 to retrieve a particular selection of RT records.

RT objects received from the RT objects database 102 may be processed by a miniaturization agent 111. In various embodiments, the miniaturization agent 111 may reduce the size and complexity of RT datasets by using parsing logic 112 to parse, organize, and articulate RT objects into compact RT datasets 114 including miniature RT objects. Using a coder/decoder 113, the miniaturization agent 111 may decode RT objects to perform processing operations and/or encode compact RT datasets 114 into a standardized format (e.g., DICOM, and the like) compatible with a wide variety of RT treatment planning systems and other computer systems processing medical information.

In various embodiments, parsing logic 112 includes instructions for navigating the network of internal and/or external links between objects included in an RT dataset and simplifying and reducing the size of RT datasets. In various embodiments, the network of external links may be a hierarchical network of links having four tiers—patients, studies, series, and images. For example, all RT datasets and/or objects having a patient ID belong to particular patient; all RT datasets and/or objects having the patient ID and the study ID are included in a particular study corresponding to a course of treatments; all RT datasets and/or objects having the patient ID, the study ID and the series ID are included in a particular Series corresponding to an individual treatment; and all RT datasets and/or objects having the patient ID, the study ID, the series ID, and the image ID are included in a particular image or image component. By navigating the hierarchical network of links, parsing logic 112 may efficiently locate the RT objects required to render a RT dataset for a particular patient, treatment, and/or study. In addition to the hierarchical network of external links between objects and/or groups of objects, each individual object may include a network of internal links that associates each component of an object required to display a particular aspect of an RT dataset.

Once all of the objects are located using the network of external links, parsing logic 112 may then navigate the network of internal links within each object to collect only the subset of information needed to display one or more images, graphs, visualizations, and other aspects of an RT dataset. For example, the parsing logic 112 may export and extract only the standard DICOM patient-specific objects and/or object components having the correct patient, study, series, and/or image ID from a RT planning system. The parsing logic 112 may then separate the aspects of RT object data required to view the RT dataset (i.e., viewing data) from a large amount of radiation delivery machine-specific physics data and models and computational algorithm modules that are needed to create RT plans (i.e., plan creation data). Additionally, parsing logic 112 may separate the RT object data required to view the RT dataset (i.e., viewing data) from a large number of intermediate files used in the process of making RT plans (i.e., plan creation data). The viewing data may include image data, contour data, delivery beam data, radiation dose data, and other data required to display the one or more radiation therapy records. By separating and omitting large physics data, model files, computation algorithm modules, intermediate files, and other plan creation data from the RT objects, the parsing logic 112 articulates comprehensive and compact sets of miniature RT objects that maybe be assembled into the compact RT datasets 114. In various embodiments, a miniature viewer 115 may be embedded with the compact RT datasets 114 and written to a portable storage medium to enable a remote computer to access and view the compact RT datasets 114.

In various embodiments, parsing logic 112 may include instructions for compressing RT objects and/or datasets using one or more lossy or lossless compression algorithms. For example, image objects may be compressed and/or fused with similar images and/or overlaid information included in an RT object to reduce the amount of image data included in the compact RT datasets 114. Image objects not required to display the RT datasets may also be removed to reduce the amount of image data included in the compact RT datasets 114. Parsing logic 112 can include instructions for identifying and removing null, repeated, or unused attributes and/or fields of RT objects. For example, parsing logic 112 may review an RT plan object and determine the collimator filter and treatment table angle fields are null (i.e., unused and/or set to no value). Parsing logic 112 may then delete the collimator filter and treatment table angle fields from the RT system configuration attribute to the reduce the size of the configuration attribute and the RT Plan object and assemble the compact RT dataset 114 with the reduced size version of the RT Plan object (i.e., a miniature RT Plan object). Other fields and/or attributes that do not affect the visual appearance of an RT dataset may also be removed to reduce the size of the miniature RT objects included in the compact RT datasets 114. Parsing logic 112 may also include instructions for converting image data, patient records, and/or other types of data included in an RT dataset into a more efficient storage format (e.g., converting from a relational database to unstructured storage) that may be included in the compact RT datasets 114.

As shown in FIG. 1, the miniaturization agent 111 can include an encoder/decoder 113 including instructions for accessing and converting between one or more RT dataset and/or object formats. In various embodiments, the encoder/decoder 113 may reformat RT datasets and/or objects 102 received from treatment planning systems. For example, the coder/decoder 113 may decode RT objects 112 and/or datasets received from one or more treatment planning systems as a pre-requisite step to processing by parsing logic 112. This decoding step ensures the parsing logic 112 is able to articulate the compact RT datasets 114 from RT objects 102 received from any treatment planning system.

Post miniaturization by parsing logic 112, the coder/decoder 113 may encode the compact RT datasets 114 and/or miniature RT objects into a standardized format compatible with any treatment planning system 130 and/or other remote computer systems analyzing medical images. In various embodiments, encoding applied by the coder/decoder 113 may allow the compact RT datasets 114 and/or miniature RT objects to be imported into a treatment planning system so that they may be used to plan new treatments. By enabling new providers to import archived treatment planning information from previously generated RT datasets, the coder/decoder 113 of the miniaturization agent 111 reduces the amount of imaging and other diagnostic work required to begin planning new treatments for patients. Importing previously generated RT datasets into the treatment planning system used by a new provider also ensures comprehensive dose buildup and treatment records are considered when planning new treatments to reduce the risk of radiation toxicity from over exposure. In various embodiments, the coder/decoder 113 encodes the miniature RT objects as DICOM-RT objects having RT specific extensions that model Radiation Therapy practice. RT specific extensions encoded by the coder/decoder 113 can include RT Images; RT Records; RT Structure sets including contours segmenting normal tissue, organs at risk, disease sites (e.g., tumor structures), and other groupings of anatomical structures; RT Plans including include dose prescription attributes, delivery device configurations and navigation instructions, and patient setup information; and RT Dose objects including dose build-up information (e.g., volumetric dose distribution, dose volume histograms, and the like).

The coder/decoder 113 may write the compact RT datasets 114 and/or miniature RT objects to a database included in the RT achieving system 110. In various embodiments, the coder/decoder 113 may encrypt one or more of the compact RT datasets 114 and/or one or more database instances storing the compact RT datasets 114 using an encryption algorithm that complies with an encryption standard (e.g., data encryption standard (DES), advanced encryption standard (AES), etc.). Encryption algorithms used by the coder/decoder 113 may include public key encryption algorithms, private key encryption algorithms, symmetric encryption algorithms, and/or asymmetric encryption algorithms. In various embodiments, encryption performed by the coder/decoder 113 may be decrypted using a password or access key. For example, a user may decrypt the encrypted compact RT datasets 114 written on a portable storage medium by entering the correct password into a free form text field that may appear when initializing the miniature viewer 115 or otherwise accessing and/or viewing the compact RT datasets 114 provided by the miniaturization agent 111. The compact RT datasets 114 may be stored in various structured data format including, for example, as a flat file, indexed file, hierarchical database, relational database, object database, graph database, and/or any other storage mechanism. In various embodiments, the database storing the compact RT datasets 114 is indexed and searchable by one or more metadata files corresponding to a compact RT dataset 114 and/or miniature RT object (e.g., patient id, study id, series id, image id, record creation date, dataset or object size, number type of RT object modality, types of RT modalities included in a dataset, and the like).

To make the compact RT datasets 114 viewable, a miniature viewer 115 may also be included in the RT archiving system 110. In various embodiments, the miniature viewer 115 includes formatting logic 116 for assembling the miniature RT objects as viewable RT dataset representations (e.g., raster graphics, planning images having segmented contours and other images, graphs, and the like) on a personal computer 150, treatment planning system 130, and/or other remote computer. The miniature viewer 115 may also include a rendering engine 117 for downloading the compact RT datasets 114 and displaying miniature RT objects and other viewable representations of RT datasets on personal computer 150, treatment planning system 130, and/or other remote computer. In various embodiments, the object viewer 115 is implemented as compact executable file that may be embedded with the compact RT datasets 114.

In various embodiments, the formatting logic 116 can include instructions for representing data included in the compact RT datasets 114 in a visual form compatible with a personal computer 150 and/or treatment planning device 130. Formatting logic 116 may access an itemized directory object to more efficiently display the compact RT datasets 114 on a remote computer. The directory object may include a network of links between the miniature RT objects and object components included in the compact RT datasets 114. By navigating the itemized directory objects, the miniature viewer 115 may efficiently aggregate all of the objects and object components required to render a particular miniature object or other visual representation of an compact RT dataset. The miniature viewer 115 may also check the data integrity of the compact RT dataset by verifying every RT object required visualize the one or more RT objects is included in the compact RT dataset.

Formatting logic 116 can also include instructions for detecting the type of computer system accessing the compact RT datasets 114 and the computer system's available memory, storage, and/or processing capacity. After retrieving the available capacity and processing resources, formatting logic 116 may limit and/or expand the available options for viewing, importing, and/or storing the compact RT datasets 114 and/or miniature RT objects. In various embodiments, formatting logic 116 can include instructions for converting the compact RT datasets 114 to a format compatible with a media player native to a personal computer 150, a treatment planning system 130, and/or other remote computer.

To display visual aspects of miniature RT objects assembled by the formatting logic 116, the miniature viewer 115 includes a rendering engine 117 executable by a shader and/or other graphics processor. In various embodiments, the miniature viewer 115 may be implemented as a C++ application that uses one or more OpenGL or ActiveX graphic libraries for rendering functionality. Using one or more OpenGL or ActiveX libraries, the rendering engine 117 may render one or more RT datasets as raster graphics, segmented contours, multi-leaf collimator (MLC) leaves, beam routes, and other visual aspects overlaid or otherwise displayed on reference images. In various embodiments, the miniature viewer 115 may include a copy of one or more OpenGL or ActiveX libraries so that the miniature viewer 115 may display the compact RT datasets 114 regardless of whether the required OpenGL or ActiveX libraries are installed on the personal computer 150, treatment planning system 130, or other remote computer executing the miniature viewer 115. In various embodiments, to display the compact RT datasets 114, the rendering engine 117 may render color data for each image pixel on a corresponding display pixel within a display screen on the remote computer system.

In various embodiments, the rendering engine 117 can include instructions for aligning visual representations of two or more visual aspects of a particular compact RT dataset 114 generated by the formatting logic 116. For example, dose and/or beam planning information may be overlaid over a structure set contours and an anatomical image (e.g., CT Scan) to create a miniature RT Plan object that visualizes the location of isodose lines describing dose build-up in one or more regions of interest. Structure set contours and diagnostic information may be overlaid over an anatomical image to create a miniature RT Structure set object illustrating regions of interest including tumors and normal structures (e.g., organs at risk). Delivery device beam routes and other RT planning information may be overlaid over RT Structure set contours and a reference image to create a miniature RT Plan object. Alignment instructions provided by the rendering engine 117 may correct alignment errors between two or more visual aspects of miniature RT objects included in the compact RT datasets 114.

The rendering engine 117 may also correct distortion and/or resolve clarity issues in RT dataset visualizations. In various embodiments, the rendering engine 117 may include instructions for smoothing visual representations and/or resolving resolution differences between two or more visual layers included in a particular miniature RT object. To provide additional information about the quality and accuracy of the RT planning information included in the compact RT datasets 114 and/or miniature RT objects presented during treatment planning, the rendering engine 117 may include instructions for signaling when alignment corrections are made, resolution and/or distortion errors are resolved, and/or when there is an display error that cannot be resolved or corrected by the rendering engine 117.

As shown in FIG. 1, the compact RT datasets 114 and a miniature viewer 115 may be provided to an archiving module 118. In various embodiments, the archiving module 118 embeds an instance of the object viewer 115 with one or more compact RT datasets 114 to create images, visualization, graphs and other viewable miniature RT objects. The archiving module 118 may also create an itemized directory object summarizing the contents of each compact RT dataset (e.g., the objects and/or object components included in the dataset). The itemized directory object may also include a network of links specific to the particular compact RT dataset that describes how objects and object components required to display the compact RT dataset and/or a visual representation of one or more aspects of the RT dataset are related. In various embodiments, the miniature viewer 115 may navigate the network of links included in the itemized directory object to assemble and/or display the compact RT datasets 114. The archiving module 118 may then write compact RT datasets 114, the miniature viewer 115, and the directory object to a portable storage medium 120.

In various embodiments, a portable storage medium 120 may include a magnetic disk or tape, optical disk (e.g., CD-ROM or DVD), flash memory, solid-state disk (SSD), electronic random access memory (RAM), micro-electromechanical and/or any other similar media adapted to store information. A portable storage medium 120 is a stand-alone device, a cloud storage device, and/or a remote computer. Writing the compact RT datasets 114, the miniature viewer 115, and the directory object to a portable storage medium 120, reduces bulky RT treatment planning files and records into a comprehensive, compact, and viewable record of previous treatment plans that is viewable from any computer system. By embedding the miniature viewer 115 and the directory object with the compact RT datasets 114, the archiving module 118 allows the portable storage medium 120 to function independently and display the compact RT datasets 114 without requiring any specialized treatment planning software.

In various embodiments, the RT record archiving system 110 may transfer the compact RT datasets 114 directly to a personal computer 150, treatment planning system 130, or other remote computer. In various embodiments, the compact RT datasets 114 are transferred directly to a treatment planning system 130 by writing the compact RT datasets 114 to a secure cloud storage instance (e.g., an encrypted cloud server); sending a message to a treatment planning system 130 indicating the compact RT datasets 114 are ready for download; accessing a secure cloud storage instance by the treatment planning system 130; and downloading one or more compact RT datasets 114 from the cloud storage instance to the treatment planning system 130. Optionally, the message to the treatment planning system 130 may include a private key; access code; password or other authentication and/or decryption information to enhance security of the compact RT datasets 114. In various embodiments, the compact RT datasets 114 may be stored on the cloud storage instance as encrypted files that must be decrypted using an access key and/or password before they may be accessed.

In various embodiments, compact RT datasets 114 may include treatment planning information and other information that may be useful in planning new treatments. For example, historical dose build-up and other toxicity information, the effect of previous treatment on a tumor site, calibration settings, navigation routes, and shaping instructions for operating a treatment delivery device (e.g., a photon generating LINAC), and the like. In various embodiments, treatment planning information may be imported from the compact RT datasets 114 into a treatment planning system 130 to inform the planning of new RT treatments. The compact RT datasets 114 and the miniature viewer 115 used to display and/or extract data from the compact RT datasets 114 may written by the archiving module 118 in a standardized file format (e.g., DICOM and the like) that is compatible with any treatment planning system 130. In various embodiments, the miniature viewer 115 may have a built-in decryption tool that can load encrypted compact RT datasets 114 and export decrypted compact RT datasets 114 to an RT planning system 130. The miniature viewer 115 may require a password or key to decrypt the files to ensure privacy and information security. Treatment planning systems 130 process treatment planning information imported from one or more compact RT datasets 114 to accurately understand the impact and effectiveness of doses administered during previous RT treatments. Archived treatment planning information may be used to develop new RT plans by helping physicians and dosimetrists to determine an RT prescription (amount, type, session length, patient setup, RT system configuration, etc.) that maximizes the therapeutic effects and avoids radiation risk and toxicity. RT plans developed from archived treatment planning information may also be used to plan radiation beam line routes followed by a photon producing linear accelerator (LINAC) of an RT system during administration of a radiation dose.

Exemplary treatment planning systems 130 may be implemented as an independent software system and/or a software system built into a RT system. Treatment planning systems 130 may generate radiation beam line routes according to archived treatment planning information imported from one or more compact RT datasets 114, for example, tumor locations and tumor reduction size and/or rate from previous treatments and or/previous dose plans; tissue type contours and critical structure contours included in structure sets; and/or patient tissue dose distribution statistics and dose build-up information included in dose statistics. Treatment planning systems 130 that may access, display, and/or import planning information from compact RT datasets generated by the RT archiving system 110 can include, for example, iPlan by BrainLAB, GammaPlan by Elekta, Pinnacle³ by Philips, Precision by Accuray, Raystation by Raysearch, and Eclipse by Varian.

FIGS. 2A-2E illustrate exemplary visualizations of compact RT datasets 114 that may be rendered by the rendering engine 117 of the miniature viewer 115. FIG. 2A illustrates a hierarchical network of links connecting RT objects and object components within a particular compact RT dataset. FIG. 2B illustrates a single beam plan illustrating the path of a radiation delivery beam at multiple delivery depths. FIG. 2C illustrates a dose plan comprising dose buildup information and a set dose distribution isodose lines overlaid over a contoured reference image and FIGS. 2D-2E illustrate dose statistics corresponding to the dose plan in shown in FIG. 2C. As shown in FIG. 2A, parsing logic 112 may navigate a hierarchical network of links 200 to organize miniature RT objects and articulate the compact RT datasets 114. The hierarchical network of links 200 may include a patient ID 202 as the top tier of the hierarchy; study ID 204 as the second tier; series ID 206 as the third tier, and image ID 208 as the fourth tier. In various embodiments, the hierarchical network of links 200 may include a greater or fewer number of tiers and each tier may have different ID types. Each ID shown in FIG. 2A may be attached to a Unique Identifier (UID) to form the hierarchical network of links 220 used to organize a particular compact RT dataset.

The patient ID 202 may be associated with one or more study IDs 204 because each patient typically has more than one course of RT treatments. Distinct study IDs 204 may be used for each course of RT treatments planned, prescribed, and/or delivered for the patient. Each study may be organized by date, anatomical region being treated, type of RT treatment, and the like. Within each study ID 204, a plurality of series IDs 206 may be listed. Each series ID 206 may include a group of objects (e.g., images, visual enhancements, and the like) sharing the same object modality. For example, reference images may be grouped together within the same CT series ID, contoured structure sets identifying organs at risk, tumor sites and other anatomical regions of interest may be grouped together within the same RTSTRCUCT series ID, dose planning information including simulated dose build-up from each radiation delivery beam across all contoured regions within an anatomical structure set may be grouped together within the same RTDOSE series ID, and delivery beam paths and other beam navigation instructions for each beam delivering radiation in a particular treatment may be grouped together within the same RTPLAN series ID.

Individual objects included in a series ID may have an image ID 208. For example, an image showing RT planning information overlaid on a lung structure set may be identified using the Lung IMRT image ID within the RTPLAN series ID. Each image ID 208 may include a plurality of image components 210. For example, the Lung IMRT image ID may include beam path information for each of the beams delivering radiation for the radiation dose specified in the RTPLAN series. In the exemplary hierarchical network of links 200 shown in FIG. 2A, four radiation beams included as image components 210 of the Lung IMRT image. In various embodiments, a network of internal links may associate each image component within an image ID 208. Parsing logic 112 navigates the hierarchical network of links 200 and/or the internal network of links to locate and export only the images, objects, and image components required to visualize complete RT datasets. The exported images, objects, and image components are then assembled into the compact RT datasets 114.

FIG. 2B illustrates a beam plan 220 for a radiation delivery beam included in an RT treatment. The beam plan 220 may include a series of MLC leaf segments 222. Each MLC segment 222 illustrates the beam shaping and the amount of energy fluence delivered by the radiation beam at a particular depth inside the patient's body. The MLC segments are shown overlaid on an expected intensity map of a region of interest (e.g., tissue site having a tumor). In various embodiments, the intensity map may be modulated by moving MLC leaves. The uneven lines of the leaf segments 222 illustrate how the radiation beam is shaped to avoid normal structures (i.e., healthy tissues) and concentrate on tumor sites 224. As shown by the difference in color in FIG. 2B, the beam intensity is much higher over the tumor site 224 than the other anatomical regions shown. To create the beam plan 220, image components and other RT planning information from an RTPLAN Series are overlaid over a reference image depicting an anatomical region of interest included in the CT Series. In various embodiments, the reference image may show depth for an anatomical region of interest vertically (e.g., along the y axis).

FIG. 2C illustrates a dose plan 240 illustrating dose buildup information for a section of the patient's chest as a result of administering an RT treatment. Dose buildup information may account for radiation delivered by one or more radiation beams. In various embodiments, dose plans 240 illustrating aggregate dose buildup information for two or more RT treatments may be articulated by the miniaturization agent and displayed using the miniature viewer. As shown in FIG. 2C, the dose plan 240 may include a structure set resembling a contour map that superimposes color coded anatomical regions of interest (e.g., normal tissues, organs at risk, diseased areas) 242 over a reference image. In various embodiments, structure sets may associate each segmented image region having a distinct characterization, value, and/or value range with a different texture (color, pattern, pigment intensity, etc.). As shown in FIG. 2C, the structure set may identify the tumor (shown in the lower left portion of the image), the lungs (the two bean shaped lobes shown on the right and left side of the image), and other anatomical regions of interest 242.

In various embodiments, the anatomical regions of interest 242 may be identified using a contour indicating the boundaries of tumor, organ, or other region of interest 242. Dose information included in the dose plan 240 may be shown relative to the regions of interest 242 to illustrate the dose buildup on each region of interest 242 that would occur after the RT treatment was administered. Dose information may be shown as a series of isodose lines 244. In various embodiments, each isodose line 244 may correspond to a particular percentage of a radiation dose (e.g., 60.00 grays (gy) of radiation). The isodose lines 244 define the boundaries of the anatomical regions receiving the specified percentage of dose build-up. For example, all of the areas included within the 60% isodose line have a dose buildup of at least 60%. In various embodiments, the isodose lines 244 can be concentric in that the isodose line corresponding to the largest percentage dose (e.g., 105%) is centered at the tumor site and the isodose lines corresponding to lower percentage doses extend gradually out from the tumor site center so that the 100% line is around the 105% line, the 90% line is around the 100% line, the 80% line is around the 90% line and so on with the isodose line having the lowest percentage (e.g., 20%) appearing the furthest from the central tumor site. To create a dose plan 240, structure sets from the RTSTRUCT Series may be combined with dose information form the RTDOSE series and overlaid over a reference image included in the CT Series.

FIGS. 2D-2E illustrate dose distribution information for a particular RT treatment. FIG. 2D is an exemplary dose distribution profile 260 indicating the non-uniform amount of radiation received by each region of interest (e.g., the tumor, organs at risk, and other tissues) during an RT treatment. The distribution profiles 260 may include one or more dose distribution lines 262 that describe the percentage of each anatomical region of interest that receives a particular amount of radiation. To generate distribution profiles 260, dose information from the RTDOSE Series may be combined with anatomical regions of interested included in the RTSTRUCT Series.

FIG. 2E illustrates a dose statistics table 280 providing additional dose information for each anatomical region of interest. The anatomical regions of interest described in the exemplary dose statistics table 280 correspond to the anatomical regions of interest described in the distribution profile 260 shown in FIG. 2D and the dose plan 240 shown in FIG. 2C. From left to right, the columns in the dose statistics table 280 include: regions of interests (ROI); color corresponding to each ROI (clr); minimum dose of radiation within the ROI (min); average dose of radiation within the ROI (mean); maximum dose of radiation within the ROI (max); uniform effective dose of radiation administered during the RT treatment (EUD); variance (i.e., standard deviation of min and max values) (std Dev); and conformity index (CI: RTOG).

In various embodiments, the RT archiving system may articulate and display miniature RT objects including dose plans 240, dose distribution profiles 260, dose statistics tables 280 illustrating the cumulative amount of radiation absorbed by a region of interest shown in an image for any combination of past or future RT treatments (e.g., all previous RT treatments, each individual RT historical and/or future treatment, a selection of historical and/or future treatments, all future RT treatments included in a RT plan), and other visual representations of RT datasets. In various embodiments, the RT dataset visualizations provided by the RT archiving system may be specific to RT treatment variables (e.g., RT dose strength, radiation field size, projected beam alignment, beam intensity, RT ion strength, size of the ion source, patient distance from the source, use of filters (e.g., collimation, flattening, etc.), radiation shielding equipment used, and the like).

FIG. 3 illustrates an exemplary method 300 of generating and viewing compact RT datasets. At 302, RT objects are received from a treatment planning system and/or other medical record system. In various embodiments, the RT archiving system may be installed on a treatment planning system to facilitate transfer of RT objects. The RT archiving system may also be implemented on a remote computer that may receive RT objects from a treatment planning device through a wired and/or wireless connection. RT objects may include one or more object modalities including reference images, RT Structure sets, RT Images, RT Plans, RT Images, RT Doses, RT Records, and other patient medical information related to one or more RT treatments. At step 304, RT objects received by the RT archiving system are parsed by parsing logic of the miniaturization agent. Parsing may include performing one or more preprocessing operations (e.g., reformatting, decoding, extracting, etc.). Parsing performed by parsing logic may also navigate a network of links connecting RT objects and image components and other aspects included in RT datasets to extract the information required to articulate and display comprehensive RT datasets. Parsing logic may organize and assemble compact RT datasets from a plurality of RT objects by aggregating content from one or more RT objects (e.g., content having common or related Study IDs, Series IDs, image IDs, and the like). In various embodiments, parsing logic may aggregate object content required to display RT datasets by analyzing a hierarchical network of links to locate content of an RT object; extracting the content from the RT object by separating the components of the RT object required for viewing the RT object and/or RT dataset (i.e., viewing data) from the intermediate files, simulation physics and modeling algorithms, and other components required for treatment planning; downloading the content for each component required to view the RT dataset (i.e., plan creation data); and/or aggregating the downloaded content in a compact RT dataset and or miniature RT object.

For example, parsing logic may locate content required for a RT dataset dose plan by navigating the hierarchical network of links to locate an RTDOSE Series, an RTPLAN Series, a CT Series, and a RTSTRUCT Series corresponding to the particular RT dose plan for a particular patient. Parsing logic may then locate the dose within the RTDOSE Series and extract the dose information (e.g., absorbed dose percentage, dose increments, and tissue location corresponding to each absorbed dose percentage); locate the planning formation (e.g., navigation instructions and system configurations) for the dose within the RTPLAN Series; locate the reference image associated to the planning formation from the CT Series; and locate the structure set (e.g., location of regions of interest and contour pattern) for the reference image from the RTSTRUCT Series. Parsing logic then downloads each of these components and may articulate a compact RT dataset including a miniature RT dose plan object using these components at 306. Instructions for assembling and/or navigating through the objects and components included in each Series may be included in an itemized directory object generated at 308.

In various embodiments, the miniaturization agent may reduce the size of RT datasets by executing one or more compression algorithms, for example, one or more lossy or lossless compression algorithms. At 310, a miniature object viewer may be embedded with the compact RT datasets. The miniature object viewer may display the miniature RT objects and other visualizations included in the compact RT datasets articulated by parsing logic at 306 on a personal computer and/or treatment planning system. At 312, an archiving module may write the compact RT datasets, embedded miniature object viewer, and the itemized directory object to a portable storage medium. In various embodiments, the compact RT datasets and embedded miniature viewer may be written as executable files that may be accessed, opened, and visualized without installing any additional software. The compact RT datasets may be written as encrypted files that may be decrypted by entering a password and/or access key into a free form text file that may appear when opening the miniature viewer to view the compact RT datasets. The portable storage medium may be used to transfer the compact RT datasets to a personal computer, treatment planning system, or other remote computer. Once the compact RT datasets are transferred to a remote computer, the miniature viewer may enable users to view the miniature RT objects and other aspects of the compact RT datasets on a remote computer device at 314.

FIG. 4 is a block diagram representation of an exemplary method of importing treatment planning information from a compact RT dataset for use in planning a new RT treatment 400. New treatment plans developed using achieved treatment planning information imported from compact RT datasets may be reviewed and/or approved by a physician and sent to an RT system to deliver the RT treatments. At 402, a treatment planning system receives a compact RT dataset including treatment planning information from a portable storage medium. In various embodiments, treatment planning information can include objects, images, graphs, data, and other records having archived treatment planning information used to plan one or more radiation treatments administered to a patient. The compact RT dataset may be transferred to a treatment planning system by exporting the compact RT dataset from a portable storage device.

At 404, the miniature viewer may use the itemized directory object to parse treatment planning information from the compact RT dataset. In various embodiments, the itemized directory significantly increases parsing efficiency thereby improving user experience and reducing the amount of processing, memory, and network resources required to view an RT dataset. In various embodiments, the miniature viewer may use the itemized directory object to separate treatment planning information from other types of information included in the RT dataset. At 406, the miniature viewer may display the treatment planning information on the treatment planning system to allow physicians, patients, and/or dosimetrists to preview the treatment planning information an understand if it is relevant and/or likely to be helpful. At 408, using the miniature viewer embedded with the compact RT dataset, treatment planning information including RT dataset visualizations, images, graphs, and other data may be exported from the portable storage medium and downloaded on the treatment planning device. Treatment planning information imported from the compact RT datasets, may be saved on the treatment planning system for further analysis.

At 410, treatment planning information included in the compact RT dataset can be reviewed on a treatment planning system and used to plan a new RT treatment. In various embodiments, treatment planning information may be imported into a treatment planning system so that simulation, planning, and modeling functionalities of the treatment planning system may be used to edit and manipulate the archived treatment planning information. For example, dose build-up information included in archived treatment planning information may be combined with dose buildup information generated from new RT treatment simulation to provide accurate historical dose build-up values that may be used to determine a patient's risk of radiation toxicity. In various embodiments, the size of the tumor site may be extracted from archived treatment planning information to better understand the effectiveness of a particular radiation dose, beam path, and/or delivery mechanism.

Functionality that may be provided by the miniature viewer to enhance compatibility with a variety of treatment planning systems can include retrieving and/or downloading treatment planning information from the compact RT dataset; storing information retrieved from the compact RT dataset in memory or storage; pushing archived treatment planning information to a treatment planning system; editing (e.g., correcting, combining, revising, etc.) archived treatment planning information before pushing it to a treatment planning system; and/or formatting and/or converting archived treatment planning information to be compatible with a treatment planning system.

Using the archived treatment planning information revived from the compact RT datasets, a treatment planning system may generate a new RT treatment plan for on-going RT treatments that accounts for historical data (e.g., cumulative radiation absorption, preview RT treatment optimizations, patient response to RT treatment, etc.) included in the viewable RT object. At step 412, the treatment planning system may provide the new RT treatment plan to a radiation delivery device (e.g., ion beam apparatus included in a RT system). In various embodiments, before and/or simultaneously with providing a new RT treatment plan to a radiation delivery device, the treatment planning system may provide the new RT treatment plan to a physician to review and/or approve.

FIG. 5 shows an illustrative computer 500 that may implement the RT archiving system and various features and processes as described herein. The computer 500 may be any electronic device that runs software applications derived from compiled instructions, including without limitation personal computers, servers, smart phones, media players, electronic tablets, game consoles, email devices, etc. In some implementations, the computer 500 may include one or more processors 502, volatile memory 504, non-volatile memory 506, and one or more peripherals 508. These components may be interconnected by one or more computer buses 510.

Processor(s) 502 may use any known processor technology, including but not limited to graphics processors and multi-core processors. Suitable processors for the execution of a program of instructions may include, by way of example, both general and special purpose microprocessors, and the sole processor or one of multiple processors or cores, of any kind of computer. Bus 510 may be any known internal or external bus technology, including but not limited to ISA, EISA, PCI, PCI Express, NuBus, USB, Serial ATA or FireWire. Volatile memory 504 may include, for example, SDRAM. Processor 502 may receive instructions and data from a read-only memory or a random access memory or both. The essential elements of a computer may include a processor for executing instructions and one or more memories for storing instructions and data.

Non-volatile memory 506 may include, by way of example, semiconductor memory devices, such as EPROM, EEPROM, and flash memory devices; magnetic disks such as internal hard disks and removable disks; magneto-optical disks; and CD-ROM and DVD-ROM disks. Non-volatile memory 506 may store various computer instructions including operating system instructions 512, communication instructions 514, application instructions 516, and application data 517. Operating system instructions 512 may include instructions for implementing an operating system (e.g., Mac OS®, Windows®, or Linux).

The operating system may be multi-user, multiprocessing, multitasking, multithreading, real-time, and the like. Communication instructions 514 may include network communications instructions, for example, software for implementing communication protocols, such as TCP/IP, HTTP, Ethernet, telephony, etc. Application instructions 516 can include instructions for importing RT objects, organizing RT objects, articulating compact RT datasets, viewing miniature RT objects and other aspects of compact RT datasets, and the like. For example, application instructions 516 may include instructions for modules to retrieve and process RT objects to generate compact RT datasets as described above in conjunction with FIG. 1. Application data 517 may correspond to data stored by the applications running on the computer 500. For example, application data may 517 may include patient medical records, image data from medical imaging procedures, RT objects, viewable RT datasets, RT object data included coder/decoder data buffers, and/or RT object data included in one or more rendering data buffers.

Peripherals 508 may be included within the computer 500 or operatively coupled to communicate with the computer 500. Peripherals 508 may include, for example, network interfaces 518, input devices 520, and storage devices 522. Network interfaces 518 may include, for example, an Ethernet or WiFi adapter for communicating over one or more wired or wireless networks. Input devices 520 may be any known input device technology, including but not limited to a keyboard (including a virtual keyboard), mouse, trackball, and touch-sensitive pad or display. Storage devices 522 may include one or more mass storage devices for storing data files; such devices include magnetic disks, such as internal hard disks and removable disks; magneto-optical disks; and optical disks.

The foregoing description is intended to convey a thorough understanding of the embodiments described by providing a number of specific exemplary embodiments and details involving organizing RT objects, articulating compact RT datasets, miniaturizing RT objects and RT datasets, viewing miniature RT objects and compact RT datasets to improve the portability and transferability of patient medical records, specifically RT treatment records and planning information. It should be appreciated, however, that the present disclosure is not limited to these specific embodiments and details, which are examples only. It is further understood that one possessing ordinary skill in the art, in light of known systems and methods, would appreciate the use of the invention for its intended purposes and benefits in any number of alternative embodiments, depending on specific design and other needs. A user device and server device are used as examples for the disclosure.

Methods described herein may represent processing that occurs within a system (e.g., system 100 of FIG. 1). The subject matter described herein can be implemented in digital electronic circuitry, or in computer software, firmware, or hardware, including the structural means disclosed in this specification and structural equivalents thereof, or in combinations of them. The subject matter described herein can be implemented as one or more computer program products, such as one or more computer programs tangibly embodied in an information carrier (e.g., in a machine-readable storage device), or embodied in a propagated signal, for execution by, or to control the operation of, data processing apparatus (e.g., a programmable processor, a computer, or multiple computers). A computer program (also known as a program, software, software application, or code) can be written in any form of programming language, including compiled or interpreted languages, and it can be deployed in any form, including as a stand-alone program or as a module, component, subroutine, or another unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this specification, including the method steps of the subject matter described herein, can be performed by one or more programmable processors executing one or more computer programs to perform functions of the subject matter described herein by operating on input data and generating output. The processes and logic flows can also be performed by, and apparatus of the subject matter described herein can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processor of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of nonvolatile memory, including, by ways of example, semiconductor memory devices, such as EPROM, EEPROM, flash memory device, or magnetic disks. The processor and the memory can be supplemented by, or incorporated in, special purpose logic circuitry.

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods, and systems for carrying out the several purposes of the disclosed subject matter. Therefore, the claims should be regarded as including such equivalent constructions insofar as they do not depart from the spirit and scope of the disclosed subject matter.

As used herein, the singular forms “a”, “an”, and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

As used herein, the terms “and/or” and “at least one of” include any and all combinations of one or more of the associated listed items.

Certain details are set forth in the foregoing description and in FIGS. 1-5 to provide a thorough understanding of various embodiments of the present invention. Other details describing well-known structures and systems often associated with RT data processing and visualization, RT datasets, treatment planning systems, RT objects, user devices, and server devices, etc., however, are not set forth below to avoid unnecessarily obscuring the description of the various embodiments of the present invention.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

1. A method for archiving radiation therapy records for transmission and review, the method comprising:
receiving a plurality of radiation therapy objects;
parsing the plurality of radiation therapy objects to articulate a compact radiation therapy dataset;
generating a directory object including a list of components included in the compact radiation therapy dataset;
embedding a viewer with the compact radiation therapy dataset, the viewer navigating the directory object to display one or more radiation therapy records included in the compact radiation therapy dataset; and
writing the compact radiation therapy dataset, the itemized directory object, and the viewer to a portable storage medium.

2. The method of claim 1, wherein the compact radiation therapy dataset comprises a radiation therapy plan including one or more miniature plan objects that visualize one or more aspects of a radiation therapy treatment.

3. The method of claim 2, wherein the miniature plan objects include radiation beam delivery plans, dose plans, dose distribution profiles, and dose statistics.

4. The method of claim 2, comprising: the parsing the plurality of radiation therapy objects comprising:
analyzing a hierarchical network of links to locate the plurality of radiation therapy objects having viewing data included in the radiation therapy dataset;
for each radiation therapy object included in the plurality of radiation therapy objects, separating the viewing data from plan creation data included in the radiation therapy object;
downloading the viewing data as the one or more miniature plan objects; and
organizing the one or more miniature plan objects as the compact radiation therapy dataset.

5. The method of claim 4, wherein viewing data includes image data, contour data, delivery beam data, radiation dose data, and other data required to display the one or more radiation therapy records.

6. The method of claim 4, wherein plan creation data includes treatment simulation physics and modeling algorithms, radiation delivery machine-specific physics data and models, computational algorithm modules for creating radiation therapy plans, and intermediate files used in the process of making radiation therapy plans.

7. The method of claim 4, wherein the hierarchical network of links comprises: patient IDs, study IDs, series IDs, and image IDs.

8. The method of claim 1, wherein the viewer comprises a miniaturized executable file having a few dynamically loadable graphics libraries.

9. The method of claim 1, wherein the viewer presents the compact radiation therapy dataset in a hierarchy that is defined in DICOM standards for radiation therapy practice.

10. The method of claim 2, comprising the viewer navigating the itemized directory object to display one or more radiation therapy records comprising:
parsing the directory object to aggregate multiple miniature radiation therapy objects into a plan object, wherein the directory object includes a network of external links between the multiple miniature radiation therapy objects and the plan object;
checking data integrity of the compact radiation therapy dataset by verifying every miniature radiation therapy object required visualize the plan object is included in the compact radiation therapy dataset; and
rendering, by the viewer, the plan object using one or more graphics libraries.

11. The method of claim 10, wherein the directory object further comprises a network of internal links between the components of the compact radiation therapy dataset included in each miniature radiation therapy object and one or more of the multiple miniature radiation therapy objects.

12. The method of claim 10, wherein the viewer renders the plan object on a laptop, smartphone, tablet, or other personal computer device.

13. A method for planning radiation therapy treatments comprising:
receiving a compact radiation therapy dataset and a viewer from a portable storage medium, wherein the compact radiation therapy dataset includes a radiation therapy plan having one or more miniature plan objects that visualize one or more aspects of a radiation therapy treatment;
displaying, by the viewer, the one or more miniature plan objects, wherein the one or more miniature plan objects include treatment planning information;
evaluating the treatment planning information;
importing the treatment planning information into a treatment planning system; and
generating a new radiation therapy plan including one or more new radiation therapy treatments using the treatment planning information.

14. The method of claim 13, wherein the one or more miniature plan objects comprise planning images, segmented structure sets, 3D dose objects, and radiation therapy plan objects.

15. The method of claim 13, wherein treatment planning information comprises image data, contour data, dose data, and delivery device data collected for the radiation therapy treatment plan.

16. The method of claim 14 wherein the evaluating the treatment planning information comprises:
viewing a 3D dose object including dose distributions and segmented structure sets displayed on a planning image, wherein the segmented structure sets include one or more treated target areas and nearby normal structures;
generating dose statistics of the treated target areas and the nearby normal structures; and
determining a patient's radiation exposure based on the dose statistics.

17. The method of claim 16, wherein dose statistics include isodose lines describing an amount of radiation absorbed by the treated target areas and the nearby normal structures within the isodose lines.

18. The method of claim 13, comprising:
transmitting the new radiation therapy plan to a radiation delivery device to administer the new radiation therapy treatment to a patient.

19. The method of claim 13, wherein the new radiation therapy plan comprises: radiation dose prescriptions, plan delivery parameters, and instructions for operating a radiation treatment delivery device.

20. A radiation therapy archiving system comprising:
a miniaturization agent receiving radiation therapy objects from an object database, wherein the miniaturization agent includes parsing logic and a coder/decoder for generating compact radiation therapy datasets,
the parsing logic parsing the radiation therapy objects to articulate a compact radiation therapy dataset including one or more miniature plan objects that visualize one or more aspects of a radiation therapy treatment, and
the coder/decoder formatting radiation therapy objects for processing by the parsing logic and standardizing the format of the one or more miniature plan objects in a hierarchy that is defined in DICOM standards for radiation therapy practice;
a database storing the compact radiation therapy datasets generated by the miniaturization agent;
a miniature viewer including a miniaturized executable file having one or more dynamically loadable files that display the one or more miniature plan objects included in the compact radiation therapy dataset; and
an archiving module generating a directory object including a network of links associating aspects of the multiple miniature radiation therapy objects included in the one or more miniature plan objects, wherein the archiving module writes the directory object, the compact radiation therapy dataset, and the miniature viewer to a portable display medium.
